(19) United States
(12) Patent Application Publication
(10) Pub. No.: US 2009/0208592 A1
(43) Pub. Date: Aug. 20, 2009

(54) THICKENED PRODUCT AND METHOD FOR TRANSFERRING HEATED FLOWABLE COSMETIC ONTO SKIN

(75) Inventor: Stephen Roy Barrow, Trumbull, CT (US)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., D/B/A UNILEVER, Englewood Cliffs, NJ (US)

(21) Appl. No.: 12/107,155

(22) Filed: Apr. 22, 2008

Related U.S. Application Data
(60) Provisional application No. 61/029,400, filed on Feb. 18, 2008.

(51) Int. Cl. A61K 33/12; A61K 33/00; A61P 17/00

(52) U.S. Cl. 424/683; 424/600

(57) ABSTRACT

A product and method for treating skin with a heated lotion is herein described. The product is a package with associated instructions for applying a heated cosmetic composition to skin dispensed from a heating device. The cosmetic composition includes an inorganic structurant material such as a clay which functions to retain viscosity over a wide temperature range and even under shear allows clean cut-off of product at a nozzle of the heating device.

### BACKGROUND OF THE INVENTION

1. Field of the Invention

The invention concerns a cosmetic product that includes a flowable thickened cosmetic composition dispensed from a heating device wherein the composition retains viscosity over a wide temperature range and even under shear to provide a clean cut-off from a dispensing nozzle or orifice.

2. The Related Art

Heated lotions have several benefits. A fleeting unpleasant wince may occur when a cold fluid is first topically applied to human skin. Warmed lotions do not elicit the same negative response.

Warmed cosmetic fluids are believed to better penetrate the skin. Warmth is viewed as an assistance in opening pores. This allows deeper penetration into the skin of the cosmetic fluid.

Therapeutic effects may also be achievable by heated lotions. These may mitigate joint aches, sore muscles and other body tightness. For all these reasons, mechanical devices have been developed to heat cosmetic fluids.

U.S. Pat. No. 6,216,911 B1 (Kreitemier et al.) describes an apparatus for quickly heating a predetermined volume of viscous fluid. The fluid is then efficiently dispensed at one or more selected temperatures. In one embodiment, the predetermined volume of viscous fluid is partially housed in a predelivery chamber separate from the main fluid reservoir. An apparatus described by this patent is commercially available from New Sensations LLC, Englewood, Colo. under the brand New Sensation Lotion Spa.

U.S. Patent Application Publication 2002/0108965 A1 (Hill et al.) discloses a fluid heating and dispensing device with a first reservoir, a second reservoir, a pump, a heating device and a delivery device. This document appears to describe a commercially available apparatus from Conair Corporation under the designation HLD 31 and HLD 20.

U.S. Pat. No. 6,056,160 (Carlucci et al.) reports a device for heating and dispensing, to a user through an outlet, a foaming liquid such as shaving cream from a pressurized can.

With the advent of suitable delivery devices, a need has developed to improve the cosmetic fluids dispensed therefrom. Heating thins out most fluids. Therefore it is necessary to provide a thickening system. It has been difficult to identify appropriate thickeners. Not only must these materials viscosify but they must also produce a composition with a clean cut-off from a dispensing nozzle or outlet orifice. Still further, the thickening system must not clog the nozzle or orifice outlet.

### SUMMARY OF THE INVENTION

A cosmetic product for use with a heating device is provided which includes:
(i) a cosmetic composition having from about 0.1 to about 20% by weight of an inorganic structurant material formed as layered platelets in a cosmetically acceptable carrier;
(ii) a package containing the cosmetic composition; and
(iii) instructions associated with the package describing use of the cosmetic composition which includes charging the cosmetic composition into the heating device, applying heat to the composition, thereafter activating a dispensing mechanism associated with the device and transferring dispensed heated composition to a human body.

Further, a method for treating skin is provided which includes:
(i) providing a cosmetic composition including from about 0.1 to about 20% by weight of an inorganic structurant material formed as layered platelets in a cosmetically acceptable carrier;
(ii) providing a heating device including a chamber for receiving the cosmetic composition, a heating element for imparting heat to the cosmetic composition, and an outlet for dispensing heated cosmetic composition; and
(iii) dispensing from the device and applying the heated cosmetic composition onto the skin.

### DETAILED DESCRIPTION OF THE INVENTION

Now it has been found that inorganic structurant materials formed as layered platelets such as found in many clays are very useful in overcoming problems in the dispensing of heated flowable cosmetic compositions. The inorganic structurant materials allow dispensed heated cosmetic composition to maintain an even viscosity over a temperature gradient. Good shear stress properties of the structurant materials also provides for clean cut-off of composition from the exit nozzle.

Dispensing devices for heating cosmetic fluid compositions as noted above have been described in U.S. Pat. No. 6,216,911 B1; U.S. Patent Application Publication 2002/0108965 A1 and U.S. Pat. No. 6,056,160, the specifications of which are herein incorporated by reference. Also there are commercial devices available. One device is sold by Conair Corporation of Stamford, Conn. and another by New Sensations LLC of Englewood, Colo.

Heating devices of the present invention are best operated to deliver a composition that exhibits a dispensed temperature between about 30° to about 60° C., more preferably from 38° C. to 54° C., even more preferably from 40° to 49° C. and optimally from 42° to 46° C.

Inorganic structurant materials according to the present invention are in layered platelet form. Clays are particularly suitable. Clays are composed of extremely fine particles of clay minerals which are layer-type alumino silicates containing structural hydroxyl groups. In some clays iron or magnesium substitutes for aluminum in the lattice. Varieties of clays include kaolinites, bentonites, illites, montmorillonites, attapulgites and smectites. Bentonites are native hydrated colloidal aluminum silicate clays available from ECC America under the tradename Bentonite H and from Whittaker, Clark and Daniels under the tradename Mineral Colloid BP 2430®. Hectorite is one of the montmorillionite minerals that is a principle constituent of bentonite clay. Hectorite is available from Rheox Inc. under the tradename Bentone EW® and Macaloid®. Synthetic sodium magnesium silicate clays may also have use. Also of use is Volclay® NF which is a high purity air-classified sodium bentonite with an average dry particle size smaller than 74 microns. Most preferred are modified magnesium aluminum silicate materials sold under the tradename Veegum® by the R.T. Vanderbilt Company, Inc., Norwalk, Conn.

Clay embodiments of the present invention advantageously have average dry particle size ranging from about 1 to about 5,000 micron, preferably from about 40 to about 1,000 micron, more preferably from about 30 to about 1000 micron, most especially from 80 to 800 micron.

Amounts of the inorganic structurant material may range from about 0.1 to about 20%, preferably from about 0.5 to about 10%, more preferably from about 0.8 to about 2%, and optimally from 1 to 1.5% by weight of the composition.

Compositions of the present invention will also include a cosmetically acceptable carrier. Water is the most preferred carrier. Amounts of water may range from about 1 to about 99%, preferably from about 5 to about 90%, more preferably from about 35 to about 70%, optimally between about 40 and about 60% by weight. Ordinarily the compositions will be water and oil emulsions of the W/O or O/W variety.

Other cosmetically acceptable carriers may include mineral oils, silicone oils, synthetic or natural esters, fatty acids and alcohols and humectants. Amounts of these materials may range from about 0.1 to about 50%, preferably from about 0.1 to about 30%, more preferably from about 1 to about 20% by weight of the composition.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from about 3 to about 9, preferably from about 4 to about 5, silicon atoms.

Linear volatile silicone materials generally have viscosities less than about 5 centistokes at 25° C. while cyclic materials typically have viscosities of less than about 10 centistokes.

Nonvolatile silicone oils useful as carrier material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 100,000 centistokes at 25° C.

Among suitable esters are:
(1) Alkenyl or alkyl esters of fatty acids having 10 to 20 carbon atoms. Examples thereof include isopropyl palmitate, isopropyl isostearate, isononyl isonanonoate, oleyl myristate, oleyl stearate, and oleyl oleate.
(2) Ether-esters such as fatty acid esters of ethoxylated fatty alcohols.
(3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) Wax esters such as beeswax, spermaceti, myristyl myristate, stearyl stearate.
(5) Sterols esters, of which soya sterol and cholesterol fatty acid esters are examples thereof.

Fatty acids having from 10 to 30 carbon atoms may be included in the compositions of this invention. Illustrative of this category are pelargonic, lauric, myristic, palmitic, stearic, isostearic, hydroxystearic, oleic, linoleic, ricinoleic, arachidic, behenic and erucic acids.

Humectants of the polyhydric alcohol-type may also be included in the compositions of this invention. The humectant aids in increasing the effectiveness of the emollient, reduces scaling, stimulates removal of built-up scale and improves skin feel. Typical polyhydric alcohols include glycerol (also known as glycerin), polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably glycerin. The amount of humectant may range anywhere from 0.5 to 30%, preferably between 1 and 15% by weight of the composition.

Emulsifiers may be present in cosmetic compositions of the present invention. Total concentration of the emulsifier may range from about 0.1 to about 40%, preferably from about 1 to about 20%, optimally from about 1 to about 5% by weight of the total composition. The emulsifier may be selected from the group consisting of anionic, nonionic, cationic and amphoteric actives. Particularly preferred nonionic surfactants are those with a C₁₀-C₂₀ fatty alcohol or acid hydrophobe condensed with from about 2 to about 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; and polyoxyethylene sorbitan as well as combinations thereof. Alkyl polyglycosides and saccharide fatty amides (e.g. methyl gluconamides) are also suitable nonionic emulsifiers.

Preferred anionic emulsifiers include soap, alkyl ether sulfate and sulfonates, alkyl sulfates and sulfonates, alkylbenzene sulfonates, alkyl and dialkyl sulfosuccinates, C₈-C₂₀ acyl isethionates, C₈-C₂₀ alkyl ether phosphates, alkylethercarboxylates and combinations thereof.

Preservatives can desirably be incorporated into the cosmetic compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are iodopropynyl butyl carbamate, phenoxyethanol, methyl paraben, propyl paraben, imidazolidinyl urea, sodium dehydroacetate and benzyl alcohol. The preservatives should be selected having regard for the use of the composition and possible incompatibilities between the preservatives and other ingredients in the emulsion. Preservatives are preferably employed in amounts ranging from about 0.01% to about 2% by weight of the composition.

Additional thickening agents may be included in compositions of the present invention. Particularly useful are the polysaccharides. Examples include starches, natural/synthetic gums and cellulosics. Representative of the starches are chemically modified starches such as aluminum starch octenylsuccinate. Suitable gums include xanthan, sclerotium, pectin, karaya, arabic, agar, guar, carrageenan, alginate and combinations thereof. Suitable cellulosics include hydroxypropyl cellulose, hydroxypropyl methylcellulose, ethylcellulose and sodium carboxy methylcellulose. Synthetic polymers are still a further class of effective thickening agent. This category includes crosslinked polyacrylates such as the Carbomers, polyacrylamides such as Sepigel® 305 and taurate copolymers such as Simulgel EG® and Aristoflex® AVC, the copolymers being identified respectively by INCI nomenclature of Sodium Acrylate/Sodium Acryloyldimethyl Taurate and Acryloyl Dimethyltaurate/Vinyl Pyrrolidone Copolymer.

Amounts of the additional thickener may range from about 0.001 to about 5%, preferably from about 0.1 to about 2%, optimally from about 0.2 to about 0.5% by weight.

Colorants, fragrances and abrasives may also be included in compositions of the present invention. Each of these substances may range from about 0.05 to about 5%, preferably between 0.1 and 30% by weight.

Except in the operating and comparative examples, or where otherwise explicitly indicated, all numbers in this description indicating amounts of material ought to be understood as modified by the word “about”.

The term “comprising” is meant not to be limiting to any subsequently stated elements but rather to encompass non-specified elements of major or minor functional importance. In other words the listed steps, elements or options need not be exhaustive Whenever the words “including” or “having” are used, these terms are meant to be equivalent to “comprising” as defined above.

The following Examples will more fully illustrate the embodiments of this invention. All parts, percentages and proportions referred to herein and in the appended claims are by weight unless otherwise indicated.

### EXAMPLE 1

A typical emulsion type cosmetic composition of the present invention is reported in Table 1.

TABLE 1

| Component | Weight % |
|---|---|
| Stearic Acid | 2.34 |
| Glyceryl Monostearate/Stearamide AMP | 1.38 |
| Glycerol Monostearate | 0.65 |
| Cetyl Alcohol | 0.37 |
| Petrolatum | 1.25 |
| Isopropylmyristate | 1.30 |
| Disodium EDTA | 0.05 |
| Veegum ® | 1.00 |
| Glycerin | 10.00 |
| Simulgel EG ® | 0.75 |
| Titanium Dioxide | 0.10 |
| Triethanolamine (99%) | 0.70 |
| Glydant Plus ® | 0.09 |
| DMDM Hydantoin | 0.17 |
| Silicone 50 ct | 1.50 |
| Silicone DC 1501 ® | 0.50 |
| Ganzpearl GMP 0820 ® | 0.75 |
| Fragrance | 0.30 |
| Water | Balance |

The formula of Table 1 was formulated in the following manner. A reactor was charged with the deionized water and disodium EDTA. Heat was applied till 60° C. in combination with stirred mixing. Veegum® (only in Sample A) was added and heating continued for 10 minutes Simulgel EG® was added to the reactor and the temperature maintained at 77-80° C. for 10 to 15 minutes. In a separate vessel, the oil phase components were added. Light mixing of the batch was performed with heating in a water bath to 75-77° C. The water reactor was maintained at 60-65° C. and slow addition occurred for glycerin, titanium dioxide and triethanolamine. Continuous mixing was done until the aqueous system was uniform. Very slowly the oil phase was added to the water phase at 75-77° C. under moderate mixing. After full emulsification, the batch was agitated for a further 5 minutes. Thereupon the resultant emulsion was homogenized using an ARDE Barenco® apparatus for 20-30 seconds at 35%. The resultant system was then topped with further deionized water. Cooling then began with a large sweep (50 rpm) mixer. Preservatives Glydant Plus® and DMDM Hydantoin were then added with the batch held at 50-55° C. Thereafter a slurry of Ganzpearl GMP 0820® in the silicone oils were added to the batch. At a temperature of 45-50° C., the fragrance was charged to the reactor. Heating was then discontinued and mixing was stopped when the temperature reached 38-40° C.

The finished cosmetic composition was then charged into a Conair HLD31® Lotion Dispenser.

### EXAMPLE 2

Herein experiments are reported which determine the effect of temperature on the physical properties of a base lotion as a function of added structurant. The experiments utilized Example 1 as a base lotion except that Veegum® (magnesium aluminum silicate) was substituted at 1% with other traditional thickeners. Test procedure involved measuring the viscosity at a shear stress of 20 Pascals between the temperature of 32° and 48° C. Shear stress was chosen because it represents the force of gravity. The two temperature range limits were selected because they represent the average skin surface temperature and average temperature of a lotion dispensed from a heated lotion delivery device. Table 2 reports the percentage change in viscosity between the aforementioned skin temperature and that of the dispensed heated composition.

TABLE 2
Viscosity at 20 Pascals (mPa-sec)

| Structurant | 32° C. | 48° C. | % Change |
|---|---|---|---|
| Veegum ® (magnesium aluminum | 66860 | 175100 | 162 |
| silicate) | | | |
| Volclay ® NF | 6766000 | 1267000 | −81 |
| Carbopol ® 934* | 24900000 | 23870000 | −4 |
| Disteareth-100 IPDI** | 15680 | 7113 | −55 |
| Hydroxypropyl Methyl Cellulose | 26700 | 5731 | −79 |

*Carbopol ® 934 is a typical crosslinked polyacrylate
**Disteareth-100 IPDI is sold as Dermothix ® 100 and is an ethoxylated urethane thickener.

The results as recorded in Table 2 reveal that Veegum® not only retains viscosity at elevated temperatures but even provides for a significant increase in that viscosity. By contrast, the other structurants showed a decrease in viscosity upon reaching higher temperature. The results demonstrate the effectiveness of magnesium aluminum silicate for use with the heated lotion device of the present invention.

### EXAMPLE 3

The results from Example 2 reveal what occurs concerning flowability of a composition as temperature increases. Further experiments herein demonstrate the effect of structurants on the composition after it has been dispensed and becomes cold. Yield point values were obtained to understand the latter phenomenon. The same formulations were employed as in Example 2.

Yield point is the amount of force, or shear stress, necessary to initiate flow. A controlled stress rheometer (TA Instrument AR 2000) was used for the test. The AR 2000 is a type of instrument that enables the stress to increase from a point below the yield stress, capturing the moment when the material starts to flow.

The procedure is done at 25° C. using a 4 cm 2% stainless steel cone. Stress is ramped from 0.0 to 50 Pascals in two minutes. The ramp is stopped when there is a strain value of 20 (dimensionless). This indicates that the product has started to flow. A computer is utilized to determine the amount of stress or yield point in Pascals. Table 3 reports the results.

TABLE 3

| | Yield Point (Pa) | | |
|---|---|---|---|
| Structurant | 25° C. | 45° C. | % Change |
| Veegum ® | 41.9 | 40.8 | −2.5 |
| Volclay ® NF | 35.0 | 34.1 | −2.4 |
| Carbopol ® 934* | 73.6 | 56.4 | −23.4 |
| Disteareth-100 | 27.7 | 14.5 | −47.7 |
| IPDI** | | | |
| Hydroxypropyl | 16.6 | 11.5 | −30.6 |
| Methyl Cellulose | | | |

*Carbopol ® 934 is a typical crosslinked polyacrylate
**Disteareth-100 IPDI is sold as Dermothix ® 100 and is an ethoxylated urethane thickener.

The results indicate that both Veegum® and Volclay® NF are the best structurants for stability purposes. Viscosity changes only minimally as temperature increases by 20°. By contrast, Carbopol® 934 (crosslinked polyacrylate), Disteareth-100 IPDI (ethoxylated urethane) and hydroxypropyl methylcellulose all exhibited significant double digit declines in the yield point.

What is claimed is:

1. A cosmetic product for use with a heating device comprising:
(i) a cosmetic composition comprising from about 0.1 to about 20% by weight of an inorganic structurant material formed as layered platelets in a cosmetically acceptable carrier;
(ii) a package containing the cosmetic composition; and
(iii) instructions associated with the package describing use of the cosmetic composition which includes charging the cosmetic composition into the heating device, applying heat to the composition, thereafter activating a dispensing mechanism associated with the device and transferring dispensed heated composition to a human body.

2. The product according to claim 1 wherein the inorganic structurant material is a clay.

3. The product according to claim 1 wherein the inorganic structurant material is magnesium aluminum silicate.

4. The product according to claim 1 wherein the inorganic structurant material is present in an amount from about 0.5 to about 100% by weight of the composition.

5. The product according to claim 1 wherein the inorganic structurant material is present in an amount from 1 to 1.5% by weight of the composition.

6. A method for treating skin comprising:
(i) a cosmetic composition comprising from about 0.1 to about 20% by weight of an inorganic structurant material formed as layered platelets in a cosmetically acceptable carrier;
(ii) providing a heating device comprising a chamber for receiving the cosmetic composition, a heating element for imparting heat to the cosmetic composition, and an outlet for dispensing heated cosmetic composition; and
(iii) dispensing from the device and applying the heated cosmetic composition onto the skin.

7. The method according to claim 6 wherein the inorganic structurant material is a clay.

8. The method according to claim 6 wherein the inorganic structurant material is magnesium aluminum silicate.

9. The method according to claim 6 wherein the inorganic structurant material is present in an amount from about 0.5 to about 10% by weight of the composition.

10. The method according to claim 6 wherein the inorganic structurant material is present in an amount from 1 to 0.5% by weight of the composition.

11. The method according to claim 6 wherein the cosmetic composition is contained in a package, and the method further comprises associating instructions with the package describing use of the cosmetic composition which comprises charging the cosmetic composition into the heating device, applying heat to the composition, activating a dispensing mechanism associated with the device and transferring dispensed heated composition to a human body.
